(19) United States
(12) Patent Application Publication
(10) Pub. No.: US 2004/0161757 A1
(43) Pub. Date: Aug. 19, 2004

(54) Use of acetyl-coa carboxylase for identifying compounds that have an insecticidal effect

(76) Inventors: Reiner Fischer, Monheim (DE); Eva-Maria Franken, Burlingame, CA (US); Ralf Nauen, Langenfeld (DE); Ute Teuschel, Leverkusen (DE)

Correspondence Address: Bayer CropScience, Patent Department, 100 Bayer Road, Pittsburgh, PA, 15205-9741, US

(21) Appl. No.: 10/450,224

(22) PCT Filed: Dec. 3, 2001
(86) PCT No.: PCT/EP01/14108

(30) Foreign Application Priority Data
Dec. 14, 2000 (DE) 100 62 422.7

(51) Int. Cl. C12Q 1/68; C12Q 1/48; C07H 21/04; C12N 9/10; C12N 5/06

(52) U.S. Cl. 435/6; 435/69.1; 435/193; 435/320.1; 435/348; 536/23.2; 435/15

(57) ABSTRACT

The invention relates to nucleic acids which encode insect polypeptides with the biological activity of acetyl-CoA carboxylases, to the polypeptides encoded by them, and to their use for identifying novel, insecticidally active compounds. The invention furthermore relates to methods of finding modulators of these polypeptides, and to the use of these compounds as inhibitors of insect ACCase.

The invention relates to the use of polypeptides and enzyme preparations with the biological activity of an acetyl-CoA carboxylase for identifying new, insecticidally active compounds, and to methods of finding modulators of these polypeptides.

Acetyl-CoA carboxylase (EC 6.4.1.2), hereinbelow referred to as ACCase, catalyzes the biotin-dependent carboxylation of acetyl-CoA and is the pacemaker of the de novo fatty acid biosynthesis. ACCase has three domains: the biotin-carboxyl carrier (BCC), biotin carboxylase (BCase) and carboxyltransferase (CTase). The ACCase-catalyzed reaction can be divided into two steps. In a first step, a CO₂ group is transferred from bicarbonate, by the BCase activity, to a biotin which is bonded covalently to the BCC, with ATP cleavage. In the next step, the carboxyl group activated thus is transferred to acetyl-CoA by CTase, with the formation of malonyl-CoA (Knowles J R, 1989). There are two, physiologically different, forms of ACCase. In the heteromeric form, which is found in bacteria and in the chloroplasts of plants, three domains are formed by three separate, dissociable proteins. The homomeric ACCase consists of a polypeptide chain comprising all three domains and which is found in the cytosol of plants, animals and fungi (Ke J et al., 2000). In plants and vertebrates, ACCase is regulated by a large number of mechanisms, for example allosterically by citrate, palmitoyl-CoA, by phosphorylation/dephosphorylation, by protein kinases and at the level of gene expression (Munday M R & Hemingway C J 1999; Ke J et al. 2000). No information is available on the regulation of the enzymes from insects.

A large number of genes of ACCases from plants, fungi and vertebrates have already been cloned (for example Abu-Elheiga L et al. 1994; Bailey A et al. 1995; Goffeau A et al. 1996; ACCase from the Arabidopsis thaliana Genbank AAF18638546) or applied for as patents (for example Haselkorn R & Gornicki P 1999; Somers D A 1999; Jenkins A R et al. 1992). However, an annotated sequence from insects, that is to say one which is assigned to ACCase, is as yet unknown.

Inhibitors of ACCase from plants and fungi are already known as herbicides or fungicides from a large number of biochemical papers on plants and fungi (Vahlensieck H F et al. 1994; Gronwald J W 1994). Another document describes the fungicides Soraphen A and B, which are known as ACCase inhibitors, for controlling mites, which do not belong to the order of the insects (Sutter M. et al., 1991).

The effect of inhibitors of human ACCase on insects was studied in a publication (Popham, H J R et al. (1996): Effect of a hypolipidemic agent on the growth and development of the southwestern corn borer, Diatraea grandiosella. Comp. Biochem. Physiol., C: Pharmacol., Toxicol. Endocrinol. (1996), 115 (3), 247-249); however, this paper predominantly deals with aspects of physiology.

It was therefore an object of the present invention to make available ACCase from insects, to test its suitability as target for insecticides and to provide methods for identifying insecticidal active compounds.

In the present invention, crude extracts have been obtained from a variety of instars or adults of the peach aphid Myzus persicae by homogenizing them in suitable buffers. These crude extracts were pre-purified, and the ACCase activity was determined in a radioactive enzyme assay.

It has now been demonstrated for the first time within the scope of the present invention that compounds exist which inhibit the activity of ACCase from insects, for example from Myzus persicae, in an enzyme assay. The finding that certain compounds inhibit ACCase shows that ACCase is the target of these active compounds and constitutes a target protein of insecticidally active compounds.

It is furthermore demonstrated within the present invention that in particular cyclic 1,3-dicarbonyl compounds and their enols of the formula (I)

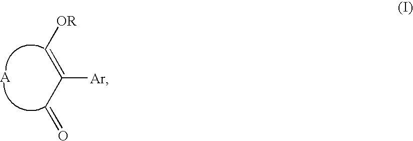

in which

Ar represents substituted aryl or hetaryl having at least one ortho-substituent,

R represents H, or represents acyl radicals, preferably the radicals COR¹ and CO₂R¹,

in which

R¹ represents optionally substituted alkyl, phenyl or hetaryl and

A together with the linked C atoms forms an optionally substituted 5- or 6-membered carbo- or heterocycle, suitable heteroatoms being, for example, N, O and/or S,

constitute ACCase inhibitors. Such cyclic 1,3-dicarbonyl compounds are known from the following documents, which are expressly incorporated into the present application:

EP-A-355 599, EP-A-377 893, EP-A415 211, EP-A-442 077, EP-A-442 073, EP-A-497 127, EP-A-501 129, EP-A-615 950, EP-A-521 334, EP-A-596 298, EP-A-613 884, EP-A-613 885, EP-A-706 527, EP-A-643 159, EP-A-741700, EP-A-668 267, EP-A-754 175, EP-A-792 272, EP-A-809 629, EP-A-825 982, EP-A-835 243, EP-A-837 847, EP-A-891 330, EP-A-912 547, EP-A-915 846, EP-A-918 775, EP-A-944 633, EP-A-1 017 674, EP-A-1 028 963, EP-A-1 056 717, WO-A-99/48869, WO-A-99/55673, EP-A-528 156, EP-A-647 637, EP-A-792 272, EP-A-799 228, EP-A-944 633, EP-A-1 017 674, EP-A-588 137, EP-A-799 228, EP-A-751 942, EP-A-588 137, EP-A-879 232, EP-A-865 438, WO-A-00/15632, WO-A-00/21946, WO-A-00/24729, EP-A-675 882, EP-A-769 001, EP-A-987 246, EP-A-773 920, EP-A-854 852, EP-A-966 420, EP-A-508 126.

It is thus shown in the present application that the abovementioned cyclic 1,3-dicarbonyl compounds can be used as inhibitors of ACCase in insects. The present invention also provides the use of the compounds of the formula (I) as inhibitors of ACCase in insects.

It is furthermore shown in the present invention that the inhibition of ACCase results in the death of treated insects. It was hitherto unknown that ACCase in insects is a target protein of insecticidally active substances. It is thus also demonstrated for the first time that ACCase is a vital enzyme for insects and therefore particularly suitable for being used as target protein in the search for further, possibly improved, insecticidal active compounds.

The present invention furthermore describes for the first time the ACCase from Drosophila melanogaster by its nucleic acid sequence, thus making it available. The nucleic acid sequence of Accession Number AAF59156 has already been available for some time. However, the meaning of the sequence, or the polypeptide encoded by it and its biological function, were hitherto unknown, as was the coding region of this sequence segment.

The meaning, function and coding region, and the polypeptide encoded by this nucleic acid, are now made available for the first time within the scope of the present invention. Thus, the cDNA of SEQ ID NO:1, which encodes the Drosophila melanogaster ACCase, and the polypeptide of SEQ ID NO:2 which is encoded by it are disclosed in the present application, and their use for identifying insecticidally and, if appropriate, also acaricidally active substances are described. The nucleic acid sequence of SEQ ID NO:1 and the polypeptide of SEQ ID NO:2 encoded by it are likewise the subject matter of the present invention.

Since the ACCases, in particular also the present ACCase from Myzus persicae and Drosophila melanogaster and from other insects show considerable homology with each other, it is also possible to use homologous polypeptides which are encoded by the relevant homologous nucleic acids, and other members of the gene family, as molecular targets of insecticidal active compounds, in particular of the compounds of the formula (I). The homologous polypeptides are especially preferably those with 60%, preferably 80%, especially preferably 90% and especially preferably 95% identity with the Myzus persicae or Drosophila melanogaster ACCase over a length of at least 20, preferably at least 25, especially preferably at least 30, consecutive amino acids and very especially preferably over the full length.

Thus, insecticidal and/or acaricidal active compounds which may be found with the aid of the ACCases according to the invention, are also capable of interacting with ACCases from a large number of other Acarina or insect species, but the interaction with the different ACCases which are found in the insects or Acarina need not always be equally pronounced. This explains, inter alia, the observed selectivity of the substances which act on this enzyme. Especially preferred ACCases, or their organisms of origin, are listed in Table 1 hereinbelow by way of example, but not by limitation:

TABLE 1

| | Preferred organisms of origin of the |
|---|---|
| | ACCases according to the invention |
| 1 | Drosophila melanogaster |
| 2 | Heliothis virescens |
| 3 | Mycus persicae |

In the present application, it is shown for the first time as exemplified by the ACCase from the peach aphid Myzus persicae that ACCases are target proteins for insecticidal active compounds and can be used for identifying new, improved insecticidal active compounds in suitable methods (assays).

The Myzus persicae and Drosophila melanogaster ACCase are particularly suitable in this context for identifying new insecticidal and, if appropriate, also acaracidal active compounds.

The present invention therefore relates to the use of insect polypeptides with the biological activity of an ACCase and to nucleic acids encoding them for identifying ACCase modulators in insects and/or Acarina, in particular of those polypeptides which have been isolated directly from insects or which are encoded by nucleic acid sequences or fragments thereof which originate from insects and which are obtained by in-vivo or in-vitro methods. The polypeptides are especially preferably those which have 60%, preferably 80%, especially preferably 90% and very especially preferably 95% identity with the Myzus persicae or Drosophila melanogaster ACCase over a length of at least 20, preferably at least 25, especially preferably at least 30, consecutive amino acids and very especially preferably over the full length.

In particular, the present invention relates to the use of ACCase from insects of the families Aphididae and Dipterea.

In particular, the present invention relates to the use of Myzus persicae ACCase and of Drosophila melanogaster ACCase as shown in SEQ ID NO: 2, and of homologous polypeptides for identifying insect ACCase modulators.

In particular, the present invention relates to the use of Myzus persicae ACCase for identifying insect ACCase modulators.

Very especially preferably, the polypeptides according to the invention thus encompass a sequence selected from

a) the sequence isolated from Mycus persicae,

b) the sequence as shown in SEQ ID NO: 2,

c) the sequence encoded by the nucleic acid of Accession Number AAF59156,

d) part-sequences of the sequences mentioned under a) to c) which retain the biological activity of an ACCase,

e) sequences which have at least 60%, preferably 80%, especially preferably 90% and very especially preferably 95% identity with the sequences mentioned under a) to d).

The degree of the identity of the amino acid sequences is preferably determined with the aid of the GAP program from the program package GCG, Version 10.0 using standard settings (Devereux et al. 1984).

The present invention also relates to the use of insect nucleic acids encoding ACCases, for identifying ACCase modulators in insects and/or Acarina.

In particular, the present invention also relates to the use of the nucleic acid encoding the Myzus persicae ACCase and of the nucleic acid encoding the Drosophila melanogaster ACCase as shown in SEQ ID NO: 1 for identifying ACCase modulators, and nucleic acid sequences which have 60%, preferably 80%, especially preferably 90% and especially preferably 95% homology therewith.

The nucleic acids according to the invention are, in particular, single-stranded or double-stranded deoxyribonucleic acids (DNA) or ribonucleic acids (RNA).

Preferred embodiments are fragments of genomic DNA which may comprise introns, and cDNAs.

The term “cDNA” as used in the present context refers to a single- or double-stranded copy of an RNA molecule and, being the copy of a biologically active RNA, is therefore free from introns, i.e. all coding regions of the gene are present in contiguous form.

The term “identity” as used in the present context refers to the number of sequence positions which are identical in an alignment. It is usually given as a percentage of the alignment length.

The term “percent (%) identity” as used in the present context when referring to a specific sequence or a specific part of the sequence is defined as the percentage of nucleotides in the nucleic acid molecule studied which is identical with the nucleotides of said specific sequence or a specific part of this sequence when the sequences are compared with each other (“alignment”) and when, if necessary, what are known as “gaps” are introduced in order to obtain the maximum percentage of identical sequences, with all parameters of the program used set to “default”.

The term “similarity” as used in the present context, in contrast, assumes the definition of a similarity metric, that is to say a measure for the desired assumed similarity between, for example, a valine and a threonine or a leucine.

The term “percentage (%) similarity”, as used in the present context, corresponds to the above-described term “percent (%) identity”, taking into consideration the conservative amino acid substitutions, in addition to the identical amino acids, when calculating the percentage.

The term “homology” as used in the present context, in turn, indicates evolutionary relationship. Two homologous proteins have developed from a shared precursor sequence. The term does not necessarily have anything to do with identity or similarity, apart from the fact that homologous sequences usually have a higher degree of similarity (or occupy more identical positions in an alignment) than non-homologous sequences.

The nucleic acids according to the invention preferably take the form of DNA or DNA fragments which correspond to genomic insect DNA, the nucleic acids preferably originating from dipterans, especially preferably from Drosophilidae.

The nucleic acids according to the invention especially preferably take the form of DNA or DNA fragments which correspond to genomic DNA of Myzus persicae or Drosophila melanogaster.

Very especially preferably, the nucleic acids according to the invention encompass a sequence selected from

a) the sequence as shown in SEQ ID NO: 1,

b) the sequence as shown in Accession Number AAF59156,

c) part-sequences of the sequences defined under a) or b) which are at least 14 base pairs in length,

d) sequences which hybridize with the sequences defined under a) or b) at a hybridization temperature of from 37° C. to 50° C.,

e) sequences which have at least 60%, preferably 80%, especially preferably 90% and very especially preferably 95% identity with the sequences defined under a) and b),

f) sequences which are complementary to the sequences defined under a) to e), and

g) sequences which, owing to the degeneracy of the genetic code, encode the same amino acid sequence as the sequences defined under a) to e).

A very especially preferred embodiment of the nucleic acids to be used in accordance with the invention is a cDNA molecule with the sequence encoding the Myzus persicae ACCase and the sequence as shown in SEQ ID NO: 1 encoding the Drosophila melanogaster ACCase.

Based on the genetic code, the nucleic acid sequence encoding the Myzus persicae ACCase can be deduced from the amino acid sequence which can be isolated as described in Example 3 and defined by means of sequencing.

Owing to the degeneracy of the genetic code, it is important to use the deduced nucleic acid sequence for verifying the nucleic acid sequence which is actually present in Myzus persicae and, if appropriate, correcting the deduced sequence, as far as this makes sense.

Isolating or verifying the genomic M. persicae sequence can be effected for example by using the primers which are derived from the deduced nucleic acid sequence and which can be utilized in PCR reactions for amplifying the target sequence by methods known to the skilled worker.

The present invention also relates to the polypeptides which are encoded by the nucleic acids according to the invention.

The term “to hybridize” as used in the present context describes the process in which a single-stranded nucleic acid molecule undergoes base pairing with a complementary strand. For example, starting from the sequence information which is mentioned herein or which can be deduced, DNA fragments can be isolated, in this manner, from insects other than Drosophila melanogaster which encode ACCases with the same or similar properties of one of the ACCases according to the invention.

Hybridization conditions are calculated approximately by the following formula:

Melting temperature Tm=81.5° C.+16.6 (log[c(Na⁺)])+0.41(% G+C)−500/n (Lottspeich & Zorbas 1998).

In this formula, c is the concentration and n the length of the hybridizing sequence segment in base pairs. For a sequence >100 bp, the term 500/n is dropped. The highest stringency involves washing at a temperature of 5-15° C. below Tm and an ionic strength of 15 mM Na⁺ (corresponds to 0.1×SSC). If an RNA sample is used for hybridization, the melting point is 10-15° C. higher.

Preferred hybridization conditions are stated hereinbelow:

Hybridization solution: DIG Easy Hyb (Roche, ZZ), hybridization temperature: 37° C. to 50° C., preferably 42° C. (DNA-DNA), 50° C. (DNA-RNA).

1. Wash step: 2×SSC, 0.1% SDS 2×5 min at room temperature;

2. Wash step: 1×SSC, 0.1% SDS 2×15 min at 50° C.; preferably 0.5×SSC, 0.1% SDS 2×15 min at 65° C.; especially preferably 0.2×SSC, 2×15 min at 68° C.

The degree of identity of the nucleic acids is preferably determined with the aid of the program NCBI BLASTN Version 2.0.4. (Altschul et al. 1997).

The term “regulatory regions” as used in the present context refers to untranslated regions of the gene in question, such as promoters, enhancers, repressor or activator binding sites or termination sequences, which interact with cellular proteins, thus governing transcription.

The present invention also relates to DNA constructs which encompass a nucleic acid to be used in accordance with the invention and a heterologous promoter.

The present invention furthermore relates to the use of such DNA constructs for identifying ACCase modulators.

The term “heterologous promoter” as used in the present context refers to a promoter with properties other than the promoter which controls the expression of the gene in question in the original organism.

The choice of heterologous promoters depends on whether prokaryotic or eukaryotic cells or cell-free systems are used for expression. Examples of heterologous promoters are the SV40, the adenovirus or the cytomegalovirus early or late promoter, the lac system, the trp system, the main operator and promoter regions of phage lambda, the control regions of the fd coat protein, the 3-phosphoglycerate kinase promoter, the acid phosphatase promoter, the Baculovirus immediate early promoter and the yeast α-mating factor promoter.

The invention furthermore relates to vectors comprising a nucleic acid according to the invention or a DNA construct according to the invention. Vectors which can be used are all those plasmids, phasmids, cosmids, YACs or artificial chromosomes which are used in molecular-biological laboratories.

The invention furthermore relates to the use of vectors comprising a nucleic acid to be used in accordance with the invention or a DNA construct to be used in accordance with the invention in methods for identifying ACCase modulators.

Vectors which can be used are all those phages, plasmids, phagmids, phasmids, cosmids, YACs, BACs, artificial chromosomes or particles suitable for particle bombardment which are used in molecular-biological laboratories.

The present invention also relates to host cells comprising a nucleic acid to be used in accordance with the invention, a DNA construct to be used in accordance with the invention or a vector to be used in accordance with the invention.

The present invention also relates to the use of such host cells for identifying ACCase modulators.

The term “host cell” as used in the present context refers to cells which do not naturally contain the nucleic acids to be used in accordance with the invention.

Suitable host cells are prokaryotic cells, such as bacteria of the genera Bacillus, Pseudomonas, Streptomyces, Streptococcus, Staphylococcus, preferably E. coli, but also eukaryotic cells such as yeasts, mammalian cells, amphibian cells, insect cells or plant cells. Preferred eukaryotic host cells are HEK 293 cells, Schneider S2 cells, Spodoptera Sf9 cells, Kc cells, CHO cells, COS1 cells, COS7 cells, HeLa cells, C127 cells, 3T3 cells or BHK cells, in particular Xenopus oocytes.

The term “polypeptides” as used in the present context refers not only to short amino acid chains which are generally referred to as peptides, oligopeptides or oligomers, but also to longer amino acid chains which are normally referred to as proteins. It encompasses amino acid chains which can be modified either by natural processes, such as post-translational processing, or by chemical prior-art methods. Such modifications may occur at various sites and repeatedly in a polypeptide, such as, for example, on the peptide backbone, on the amino acid side chain, on the amino and/or the carboxyl terminus. For example, they encompass acetylations, acylations, ADP ribosylations, amidations, covalent linkages to flavins, hem moieties, nucleotides or nucleotide derivatives, lipids or lipid derivatives or phosphatidylinositol, cyclizations, disulfide bridge formations, demethylations, cystine formations, formylations, gamma-carboxylations, glycosylations, hydroxylations, iodinations, methylations, myristoylations, oxidations, proteolytic processings, phosphorylations, selenoylations and tRNA-mediated amino acid additions.

The polypeptides according to the invention may exist in the form of “mature” proteins or as parts of larger proteins, for example as fusion proteins. They can furthermore exhibit secretion or leader sequences, pro-sequences, sequences which allow simple purification, such as polyhistidine residues, or additional stabilizing amino acids. The proteins according to the invention may also exist in the form in which they are naturally present in their organism of origin, from which they can be obtained directly, for example.

The term “complete ACCase” as used in the present context describes an ACCase which is encoded by a complete coding region of a transcription unit starting with the ATG start codon and comprising all information-bearing exon regions of the ACCase-encoding gene which is present in the organism of origin, and signals required for correct transcriptional termination.

The term “gene” as used in the present context refers to a segment from the genome of a cell which is responsible for the synthesis of a polypeptide chain.

The polypeptides according to the invention need not be complete ACCases, but may also take the form of fragments thereof, as long as they show at least the biological activity of the complete ACCase. Polypeptides from insects which exert the same type of biological activity as a Myzus persicae ACCase or Drosophila melanogaster ACCase are still considered as being in accordance with the invention. In this context, the polypeptides according to the invention need not correspond fully to the Myzus persicae or Drosophila melanogaster ACCases with regard to their sequence or catalytic activity. Polypeptides which are also considered as polypeptides according to the invention are those which are homologous to the ACCase from, for example, the following insects or to fragments thereof which retain the biological activity of ACCase:

From the order of the Isopoda, for example, Oniscus asellus, Armadillidium vulgare, Porcellio scaber.

From the order of the Diplopoda, for example, Blaniulus guttulatus.

From the order of the Chilopoda, for example, Geophilus carpophagus, Scutigera spp.

From the order of the Symphyla, for example, Scutigerella immaculata.

From the order of the Thysanura, for example, Lepisma saccharina.

From the order of the Collembola, for example, Onychiurus armatus.

From the order of the Orthoptera, for example, Acheta domesticus, Gryllotalpa spp., Locusta migratoria migratorioides, Melanoplus spp., Schistocerca gregaria.

From the order of the Blattaria, for example, Blatta orientalis, Periplaneta americana, Leucophaea maderae, Blattella germanica.

From the order of the Dermaptera, for example, Forficula auricularia.

From the order of the Isoptera, for example, Reticulitermes spp.

From the order of the Phthiraptera, for example, Pediculus humanus corporis, Haematopinus spp., Linognathus spp., Trichodectes spp., Damalinia spp.

From the order of the Thysanoptera, for example, Hercinothrips femoralis, Thrips tabaci, Thrips palmi, Frankliniella accidentalis.

From the order of the Heteroptera, for example, Eurygaster spp., Dysdercus intermedius, Piesma quadrata, Cimex lectularius, Rhodnius prolixus, Triatoma spp.

From the order of the Homoptera, for example, Aleurodes brassicae, Bemisia tabaci, Trialeurodes vaporariorum, Aphis gossypii, Brevicoryne brassicae, Cryptomyzus ribis, Aphis fabae, Aphis pomi, Eriosoma lanigerum, Hyalopterus arundinis, Phylloxera vastatrix, Pemphigus spp., Macrosiphum avenae, Myzus spp., Phorodon humuli, Rhopalosiphum padi, Empoasca spp., Euscelis bilobatus, Nephotettix cincticeps, Lecanium corni, Saissetia oleae, Laodelphax striatellus, Nilaparvata lugens, Aonidiella aurantii, Aspidiotus hederae, Pseudococcus spp., Psylla spp.

From the order of the Lepidoptera, for example, Pectinophora gossypiella, Bupalus piniarius, Cheimatobia brumata, Lithocolletis blancardella, Hyponomeuta padella, Plutella xylostella, Malacosoma neustria, Euproctis chrysorrhoea, Lymantria spp., Bucculatrix thurberiella, Phyllocnistis citrella, Agrotis spp., Euxoa spp., Feltia spp., Earias insulana, Heliothis spp., Mamestra brassicae, Panolis flammea, Spodoptera spp., Trichoplusia ni, Carpocapsa pomonella, Pieris spp., Chilo spp., Pyrausta nubilalis, Ephestia kuehniella, Galleria mellonella, Tineola bisselliella, Tinea pellionella, Hofmannophila pseudospretella, Cacoecia podana, Capua reticulana, Choristoneura fumiferana, Clysia ambiguella, Homona magnanima, Tortrix viridana, Cnaphalocerus spp., Oulema oryzae.

From the order of the Coleoptera, for example, Anobium punctatum, Rhizopertha dominica, Bruchidius obtectus, Acanthoscelides obtectus, Hylotrupes bajulus, Agelastica alni, Leptinotarsa decemlineata, Phaedon cochleariae, Diabrotica spp., Psylliodes chrysocephala, Epilachna varivestis, Atomaria spp., Oryzaephilus surinamensis, Anthonomus spp., Sitophilus spp., Otiorrhynchus sulcatus, Cosmopolites sordidus, Ceuthorrhynchus assimilis, Hypera postica, Dermestes spp., Trogoderma spp., Anthrenus spp., Attagenus spp., Lyctus spp., Meligethes aeneus, Ptinus spp., Niptus hololeucus, Gibbium psylloides, Tribolium spp., Tenebrio molitor, Agriotes spp., Conoderus spp., Melolontha melolontha, Amphimallon solstitialis, Costelytra zealandica, Lissorhoptrus oryzophilus.

From the order of the Hymenoptera, for example, Diprion spp., Hoplocampa spp., Lasius spp., Monomorium pharaonis, Vespa spp.

From the order of the Diptera, for example, Aedes spp., Anopheles spp., Culex spp., Drosophila melanogaster, Musca spp., Fannia spp., Calliphora erythrocephala, Lucilia spp., Chrysomyia spp., Cuterebra spp., Gastrophilus spp., Hyppobosca spp., Stomoxys spp., Oestrus spp., Hypoderma spp., Tabanus spp., Tannia spp., Bibio hortulanus, Oscinella frit, Phorbia spp., Pegomyia hyoscyami, Ceratitis capitata, Dacus oleae, Tipula paludosa, Hylemyia spp., Liriomyza spp.

From the order of the Siphonaptera, for example, Xenopsylla cheopis, Ceratophyllus spp.

In comparison with the corresponding regions of naturally occurring ACCases, the polypeptides according to the invention can have deletions or amino acid substitutions as long as they still exert at least one biological activity of the complete ACCases. Conservative substitutions are preferred. Such conservative substitutions encompass variations, one amino acid being replaced by another amino acid from among the following group:

1. small aliphatic residues, unpolar residues or residues of little polarity: Ala, Ser, Thr, Pro and Gly;

2. polar, negatively charged residues and their amides: Asp, Asn, Glu and Gln;

3. polar, positively charged residues: His, Arg and Lys;

4. large aliphatic unpolar residues: Met, Leu, Ile, Val and Cys; and

5. aromatic residues: Phe, Tyr and Trp.

Preferred conservative substitutions can be seen from the following list:

| Original residue | Substitution |
|---|---|
| Ala | Gly, Ser |
| Arg | Lys |
| Asn | Gln, His |
| Asp | Glu |
| Cys | Ser |
| Gln | Asn |
| Glu | Asp |
| Gly | Ala, Pro |
| His | Asn, Gln |
| Ile | Leu, Val |
| Leu | Ile, Val |
| Lys | Arg, Gln, Glu |
| Met | Leu, Tyr, Ile |
| Phe | Met, Leu, Tyr |
| Ser | Thr |
| Thr | Ser |
| Trp | Tyr |
| Tyr | Trp, Phe |
| Val | Ile, Leu |

The present invention therefore also relates to polypeptides which exert at least the biological activity of an ACCase and which comprise an amino acid sequence with at least 60% identity, preferably 80%, especially preferably 90% identity and very especially preferably 95% identity with the Myzus persicae or the Drosophila melanogaster sequence encoded by the nucleic acid as shown in SEQ ID NO: 1, and their use for identifying ACCase modulators.

The term “biological activity of an ACCase” as used in the present context refers to the ability to catalyze the biotin-dependent carboxylation of acetyl-CoA. In this context, all three enzyme functions, i.e. the ATP-dependent elimination of a CO₂ group from bicarbonate, the biotin carrier function and the carboxylation of acetyl-CoA, or else only one or two of these reactions may be encompassed.

The nucleic acids according to the invention can be prepared in the customary manner. For example, the nucleic acid molecules in their entirety can be synthesized chemically, or else short sections of the nucleic acids according to the invention can be synthesized chemically, and such oligonucleotides can be radiolabeled or labeled with a fluorescent dye. The labeled oligonucleotides can also be used for screening cDNA libraries generated starting from insect mRNA. Clones with which the labeled oligonucleotides hybridize are chosen for isolating the DNA fragments in question. After characterization of the DNA which has been isolated, the nucleic acids according to the invention are obtained in a simple manner.

As an alternative, the nucleic acids according to the invention can be generated by means of PCR methods using chemically synthesized oligonucleotides.

The term “oligonucleotide(s)” as used in the present context refers to DNA molecules composed of 10 to 50 nucleotides, preferably 15 to 30 nucleotides. They are synthesized chemically and can be used as probes.

Moreover, host cells comprising the nucleic acids according to the invention may be cultured under suitable conditions in order to prepare the polypeptides according to the invention, in particular the polypeptide encoded by the nucleic acid sequence as shown in SEQ ID NO: 1. Thereafter, the desired polypeptides can be isolated in the customary manner from the cells or from the culture medium. As an alternative, the polypeptides may be generated in in-vitro systems.

To prepare the Myzus persicae ACCase according to the invention, a procedure may be followed in which larvae or adults are homogenized with a pestle and mortar. To this end, they may previously be frozen rapidly, for example in liquid nitrogen. The homogenate is taken up in a suitable buffer. An example of the preparation of a polypeptide according to the invention is given in Example 3.

One possible ACCase purification method is based on preparative electrophoresis, FPLC, HPLC (for example, using gel filtration columns, reversed-phase columns or mildly hydrophobic columns), gel filtration, differential precipitation, ion-exchange chromatography or affinity chromatography.

A rapid method of isolating the polypeptides according to the invention which are synthesized by host cells using a nucleic acid according to the invention starts with expressing a fusion protein, where the fusion moiety may be purified in a simple manner by affinity purification. For example, the fusion moiety may be glutathione S-transferase. The fusion protein can then be purified on a glutathione affinity column. The fusion moiety can be removed by partial proteolytic cleavage, for example at linkers between the fusion moiety and the polypeptide according to the invention which is to be purified. The linker can be designed in such a way that it includes target amino acids, such as arginine and lysine residues, which define sites for trypsin cleavage. Standard cloning methods using oligonucleotides may be employed for generating such linkers.

Other purification methods which are possible are based, in turn, on preparative electrophoresis, FPLC, HPLC (for example, using gel filtration columns, reversed-phase columns or mildly hydrophobic columns), gel filtration, differential precipitation, ion-exchange chromatography or affinity chromatography.

The terms “isolation or purification” as used in the present context mean that the polypeptides according to the invention are separated from other proteins or other macromolecules of the cell or of the tissue. The protein content of the composition containing the polypeptides according to the invention is preferably at least 10 times, especially preferably at least 100 times, higher than in a host cell preparation.

The polypeptides according to the invention may also be subjected to affinity purification without fusion moiety with the aid of antibodies which bind to the polypeptides.

The present invention also relates to methods of finding chemical compounds which bind to ACCase and/or modify its properties. Owing to the important function of ACCase, modulators which affect the activity constitute novel insecticidal and/or, if appropriate, acaricidal active ingredients. Modulators may be agonists or antagonists, or inhibitors or activators.

Owing to the property of acting as inhibitors of insect ACCase, the abovementioned cyclic 1,3-dicarbonyl compounds of the formula (I) and their enols may also be used as optionally labeled competitors in methods for finding insect ACCase inhibitors which need not belong to this group of compounds.

The term “competitor” as used in the present context refers to the property of the compounds of competing with other compounds, if appropriate compounds yet to be identified, for binding to ACCase and of displacing the former from the enzyme, or of being displaced thereby.

The term “agonist” as used in the present context refers to a molecule which accelerates or increases the ACCase activity.

The term “antagonist” as used in the present context refers to a molecule which slows down or prevents the ACCase activity.

The term “modulator” as used in the present context is a general term for agonist or antagonist. Modulators may be small organo-chemical molecules, peptides or antibodies which bind to the polypeptides according to the invention and/or modify their properties, for example their enzymatic activity. Moreover, modulators can be small organo-chemical molecules, peptides or antibodies which bind to a molecule which, in turn, binds to the polypeptides according to the invention and/or influences their biological activity. Modulators can be natural substrates and ligands, or structural or functional mimetics of these.

The modulators preferably take the form of small organo-chemical compounds.

It was demonstrated for the first time within the scope of the present invention that compounds or modulators which act on ACCase are capable of modifying the cellular processes in a manner which leads to the death of the insects treated therewith.

The present invention therefore also relates to modulators of insect ACCases which are found with the aid of a method of identifying ACCase modulators, which method is described in the present application.

The present invention furthermore encompasses methods of finding chemical compounds which modify the expression of the polypeptides according to the invention. Such “expression modulators” too may be novel insecticidal active compounds. Expression modulators can be small organo-chemical molecules, peptides or antibodies which bind to the regulatory regions of the nucleic acids encoding the polypeptides according to the invention. Moreover, expression modulators may be small organo-chemical molecules, peptides or antibodies which bind to a molecule which, in turn, binds to regulatory regions of the nucleic acids encoding the polypeptides according to the invention, thus influencing their expression. Expression modulators may also be antisense molecules.

The present invention likewise relates to the use of modulators of the polypeptides according to the invention or of expression modulators as insecticides or acaricides.

The present invention likewise relates to ACCase expression modulators which are found with the aid of the above-described method for finding expression modulators.

The methods according to the invention include high-throughput screening (HTS) and ultra-high-throughput screening (UHTS). Both host cells and cell-free preparations which comprise the nucleic acids according to the invention and/or the polypeptides according to the invention may be used.

One way of finding modulators is the incubation of a synthetic reaction mix (for example products of the in-vitro transcription) or a cellular component, such as a membrane, a compartment or any other preparation comprising the polypeptides according to the invention, together with a labeled substrate or ligand of the polypeptides in the presence and absence of a candidate molecule, which may take the form of an agonist or antagonist. The ability of the candidate molecule to increase or to inhibit the activity of polypeptides according to the invention can be seen from an increased or reduced binding of the labeled ligand or from an increased or reduced conversion rate of the labeled substrate. Molecules which bind well and which lead to increased activity of the polypeptides according to the invention are agonists. Molecules which bind well and which inhibit the biological activity of the polypeptides according to the invention are good antagonists. They may also take the form of inhibitors of the abovementioned class of insecticidal substances, but entirely novel classes of substances too may show this modulatory activity.

Modulators which reduce the activity of a polypeptide according to the invention or the expression of mRNA encoding ACCase according to the invention and/or polypeptides by at least 10%, at least 20%, at least 30%, at least 40%, at least 50%, at least 60%, at least 70%, at least 80%, at least 90% or at least 95% are suitable for use as insecticides or for being developed further to give insecticides. Such candidate molecules are then checked in further tests for toxicity to vertebrate species, such as, for example, mammals, and for their bioavailability.

Detection of the biological activity of the polypeptides according to the invention can be improved by what is known as a reporter system. In this aspect, reporter systems comprise, but are not restricted to, colorimetrically labeled or radiolabeled substrates which are converted into a product, or a reporter gene which responds to changes in the activity or the expression of the polypeptides according to the invention, or other known binding assays.

The activity of a large number of proteins, for example transmembrane proteins, can be measured advantageously in a further manner. The functional heterologous expression of such proteins in E. coli is frequently difficult or impossible. In this case, the catalytically active part of the protein can be separated by means of suitable cloning methods (for example using suitable PCR strategies), so that the gene product is a soluble protein, or a protein with better solubility, and can be purified readily. A wide range of possible methods is available for measuring the activity of soluble proteins. A particularly sensitive measurement can be carried out for example by means of fluorescence polarization using a fluorescently labeled ligand or substrate.

A further example of a method by means of which modulators of the polypeptides according to the invention can be found is a displacement assay in which the polypeptides according to the invention and a potential modulator are combined, under suitable conditions, with a molecule which is known to bind to the polypeptides according to the invention, such as a natural substrate or ligands or a substrate or ligand mimetic. An example is the abovementioned compounds of the formula (I). The polypeptides according to the invention can themselves be labeled, for example radiolabeled or calorimetrically labeled, so that the number of polypeptides which are bound to a ligand or which have undergone a conversion can be determined accurately. The efficacy of an agonist or antagonist can be determined in this manner.

Potentially insecticidal compounds which are found in one of the methods according to the invention with the aid of the nucleic acids and/or polypeptides according to the invention can be administered to the insects in a variety of ways, for example orally (see also Example 4), topically or by injection. Insecticides are frequently hydrophobic molecules and must, in such a case, usually be dissolved in organic solvents which are also capable of evaporation (for example methanol or acetone) or which are added in minor concentrations in order to facilitate uptake (ethanol, dimethyl sulfoxide).

The first step in insect experiments is, as a rule, the determination of the MLD (minimal lethal dose) following chronic exposure of the insects. Usually, the compounds are diluted and added to the feed of embryos and larvae aged 0-48 hours. In addition to the MID, this procedure also determines the percentage of eggs from which larvae still hatch, and the behavior of the larvae (movement, uptake of feed), the number of larvae which still pupate and the number of adults which they produce. Moreover, the larvae can be studied for morphological defects. After the MLD has been determined, the acute and chronic dose may be determined.

In a typical acute test, the compounds are added to the food of embryos, larvae or adults, and the insects are checked after 2 hours or following incubation overnight. In the case of embryos, the number of embryos with development defects and the percentage which survives into adulthood are determined.

In larvae, parameters which are studied are, for example, abnormal behavior, impaired movement or ecdysis. In adult animals, defects regarding the quantity or activity of enzymes, abnormal behavior and/or impaired fertility are observed.

To carry out tests for determining the chronic toxicity, the adults are placed into dishes containing the compound in question, for example for 48 hours, and they are then transferred into a clean container and the fertility of the animals or the amount of activity of a certain enzyme or the death of the insects are observed.

The examples which follow demonstrate that, surprisingly, ACCase is an essential enzyme in insects; moreover, they demonstrate that the enzyme is a suitable target protein for identifying insecticides, that it can be used in methods for identifying insecticidally active compounds and that the ACCase modulators which are identified in suitable methods can be used as insecticides. To make possible the use of ACCase, obtaining this enzyme from Myzus persicae is described by way of example, and, finally, the applicability of the present invention in the search for insecticidally active compounds is demonstrated.

### EXAMPLES

### Example 1

### Preparation of an ACCase Enzyme Preparation from Myzus persicae

Myzus persicae larvae or adults are weighed and homogenized in three times the amount of extraction buffer using a pestle and mortar. The extract is subsequently centrifuged twice for 10 minutes at 10 000 g. The supernatant contains ACCase and is used in the enzyme assay for identifying inhibitors.

The following buffer is preferably used as extraction buffer: 0.25 M sucrose, 15 mM tris/HCl pH 7.4, 4 mM EDTA, 10 mM potassium citrate (all chemicals from Sigma, St. Louis).

### Example 2

### Method for Finding Modulators

The ACCase enzyme preparation was used in a biochemical assay as described hereinbelow for finding ACCase modulators: first, an aliquot of the enzyme preparation of Example 1 was mixed with the reaction buffer and the radiolabeled substrate and the mixture was incubated. To detect the incorporation of CO₂, fuming HCl was pipetted in, and an aliquot of the reaction mixture was added dropwise to Watman filter paper and dried. The dried filter paper was transferred into scintillation tubes together with scintillation liquid. The measurement was performed in a scintillation counter (Beckman Instruments, Fullerton, USA). To screen for modulators, the test compounds were dissolved in DMSO and added to the reaction mixture together with the enzyme preparation before the first incubation step. The effect of the modulators was determined in comparison with the ACCase activity in the reaction mixture with solvent, but without modulator. FIG. 2a shows the inhibition of the Myzus persicae ACCase at different active ingredient concentrations. FIG. 2b shows the inhibition of the Myzus persicae ACCase by a range of active ingredients.

The reaction buffer used was 50 mM Tris/HCl pH 7.4, 15 mM MgCl₂, 2.5 mM ATP, 1 μg/μl bovine serum albumin, 10 mM potassium citrate, 84 mM sodium bicarbonate (all chemicals from Sigma, St. Louis).

4 mM ¹⁴C sodium bicarbonate was used as the radiolabeled substrate.

### Example 3

### Measuring the ACCase Activity for Finding ACCase Inhibitors

A part-step of the ACCase-catalyzed reaction is the fixation of the carbonate group at the cofactor biotin. This fixation takes place with the cleavage of ATP: ACCase-biotin+HCO₃⁻+ATP→ACCase-biotin-CO₂⁻+ADP+Pᵢ. To determine the ACCase activity, the phosphate being liberated is detected with the commercially available malachite green reagent. Since this takes the form of a general (unspecific) detection of phosphate, all of the materials and reagents used must be free from phosphate. The ACCase must be purified from other ATP-cleaving enzymes for this detection reaction.

(a) ACCase Preparation

Whole insects, or tissues/organs obtained from them, are comminuted in the homogenization buffer (250 mM sucrose, 50 mM tris-HCl, pH 7.4, 2 mM EDTA, 10 mM sodium citrate, proteinase inhibitor mix (Sigma P-8340), for example by comminuting in a pestle and mortar or using a homogenizer rod). The homogenate is then centrifuged to obtain a clear material. The supernatant, which contains the ACCase, is then subjected to a buffer exchange with the running buffer (50 mM Tris-HCl pH 7.4, 150 mM NaCl, 1 mM EDTA, 1 mM DTT, 0.02% NaN₃, 10% glycerol) for the further steps, using a 4 PD-10 column (Pharmacia Corporation, Peapack, N.J., USA). The crude extract is subsequently separated in a Sephacryl 26/60 S-300 column (Pharmacia Corporation, Peapack, N.J., USA) by FPLC (Pharmacia Corporation, Peapack, N.J., USA). The fractions obtained are assayed for ACCase activity as described under (b). The fractions with the highest specific ACCase activities are combined and constitute the starting materials for the inhibitor measurements described under (c) (hereinbelow referred to as ACCase solution).

(b) Activity Assay

An aliquot of the ACCase solution described under (a) is mixed with the reaction buffer (50 mM Tris-HCl pH 7.4, 20 mM MgCl₂, 1 mg/ml bovine serum albumin, 20 mM sodium citrate, 5 mM NaHCO₃, 1 mM ATP, 200 μM acetyl-CoA), and the mixture is incubated at physiological temperatures (24° C. to 37° C.). After the desired reaction time has elapsed, 2 volumes of the staining reagent (BiomolGreen (Biomol Research Laboratories Inc., Plymouth Meeting, Pa., USA), prepared as specified by the manufacturer) are added and incubation of the mixture is continued as specified by the manufacturer until the detection reaction has become apparent. To determine the unspecific background activity, reaction mixtures without the substrate acetyl-CoA are also included in the measurement, and the results are subtracted when calculating the specific ACCase activity.

(c) Method for Identifying ACCase Inhibitors

To determine the inhibition of ACCase by test substances, aliquots of the ACCase solution of (a) are mixed with the reaction buffer and the test inhibitors, and the mixture is incubated at physiological temperatures (24° C. to 37° C.). After the desired reaction time has elapsed, the detection reaction is carried out as described under (b). A control measurement without addition of inhibitor is carried out in parallel. The ACCase inhibition is then calculated in comparison with this control.

Supplier: unless specified otherwise, all chemicals are from Sigma-Aldrich Co., St. Louis, USA.

### Example 4

### Impairment of Lipid Neogenesis by ACCase Inhibitors

Peach aphid Myzus persicae larvae or adults were fed for two days with nutrient solutions with and without one of the identified ACCase inhibitors, using the sachet method (Nauen et al. 1996).

By way of example, the compound of the formula (I-A)

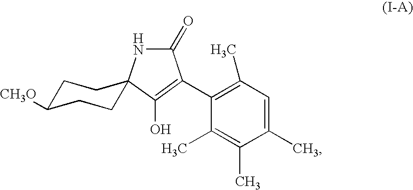

which can be referred to as 4-hydroxy-8-methoxy-3-(2,3,4,6-tetramethylphenyl)-1-azaspiro[4,5]dec-32-en-2-one, was used in this experiment.

Thereafter, the animals were collected and homogenized in organic solvent using a pestle and mortar. The organic phase was cleansed of water-soluble constituents by repeatedly extracting it by shaking with aqueous solution, and the solvent was then evaporated. The pellet which remained was taken up in a little solvent and separated by thin-layer chromatography (TLC). The lipids which were separated were stained with amido black. ¹⁴C-acetate which was incorporated into the lipids was detected by autoradiography after an exposure time of 2-3 days. To quantify the ¹⁴C-incorporation into lipids, the bands in question were scraped out after staining, dissolved, and the activity was determined in a scintillation counter. FIG. 1a shows the lipid status after acetate feeding without (lanes 1-3) and with the active ingredient of the formula (I-A) (lanes 4-6) with reference to a separated lipid extract from the peach aphid Myzus persicae. No significant differences in lipid composition and lipid content are observed. FIG. 1b shows an autoradiograph of the same TLC plate. While in lanes 1-3 those lipids into which radiolabeled acetate was incorporated during de-novo synthesis in the control aphids are discernible owing to the black color, no labeled lipids are present in the peach aphids treated with the active ingredient. Thus, no de novo lipid synthesis from acetate has taken place. FIG. 1c again shows, in the form of a diagram, how much acetate was still incorporated during de-novo synthesis in the presence of an ACCase inhibitor (0.01 ppm to 100 ppm) in comparison with the control without ACCase inhibitor. It can be seen clearly that increasing inhibitor concentrations halt the de-novo lipid biosynthesis.

The mobile phase used for the TLC chromatography was n-hexane: diethyl ether: glacial acetic acid (60:45:1).

### DESCRIPTION OF THE FIGURES

FIG. 1a)

Separated lipid extract from peach aphids Myzus persicae following acetate feeding without (lanes 1-3) and with an ACCase inhibitor (lanes 4-6). The lipids which were separated were stained with amido black.

FIG. 1b)

Autoradiograph of the TLC plate shown in FIG. 1a). ¹⁴C-acetate was detected after an exposure time of 2-3 days by means of an image. While those lipids into which radiolabeled acetate had been incorporated in de-novo synthesis in the control aphids are discernible in lanes 1-3 owing to their black color, no labeled lipids are present in the peach aphids treated with active ingredient.

FIG. 1c)

Comparison of the incorporation of ¹⁴C-acetate in the absence and presence of ACCase inhibitors (0.01 to 100 ppm) during de-novo lipid synthesis.

FIG. 2a)

Inhibition of Myzus persicae ACCase at different active ingredient concentrations. Solv=solvent (control). Comp=compound (active ingredient).

FIG. 2b)

Inhibition of Myzus persicae ACCase by different active ingredients. Comp=compound.

FIG. 3)

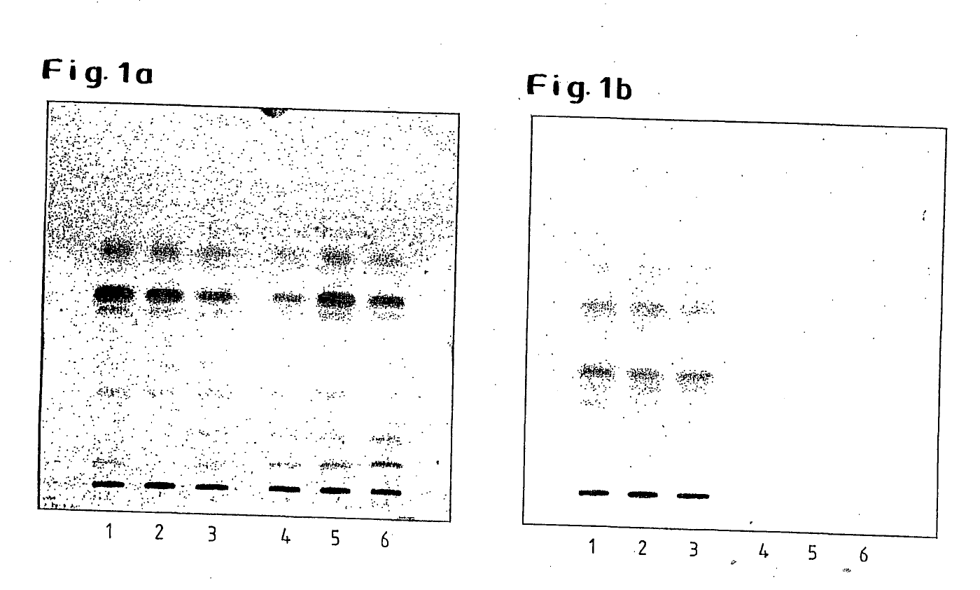

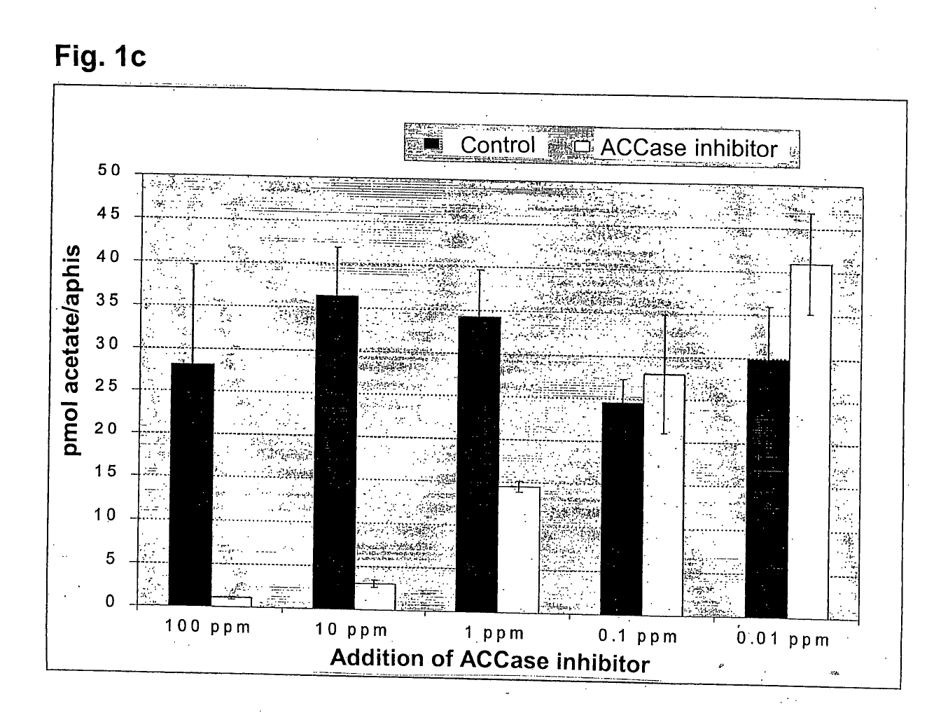

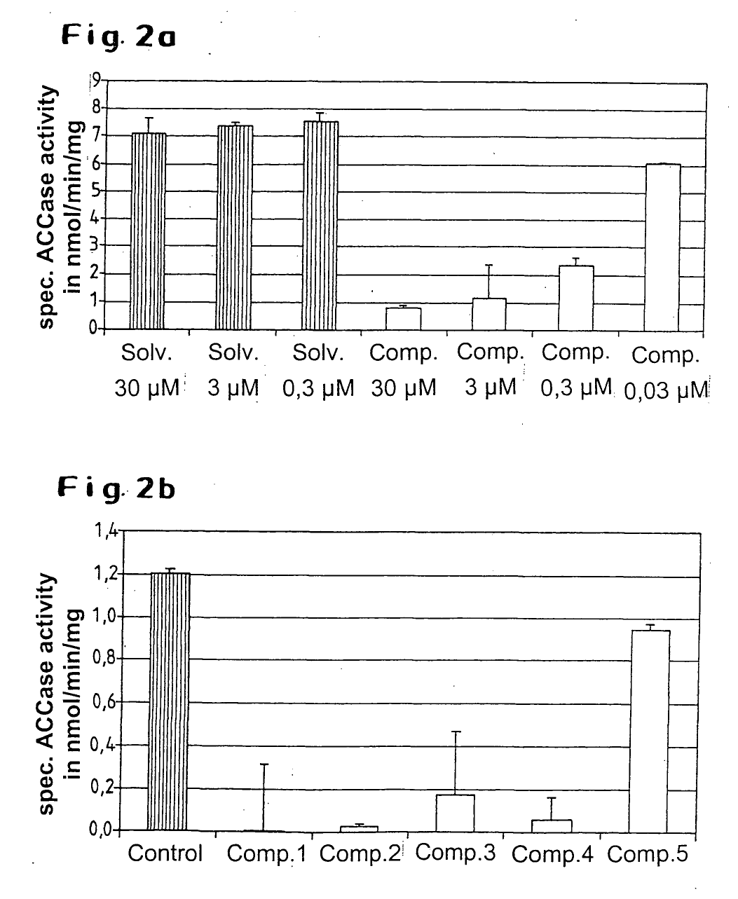

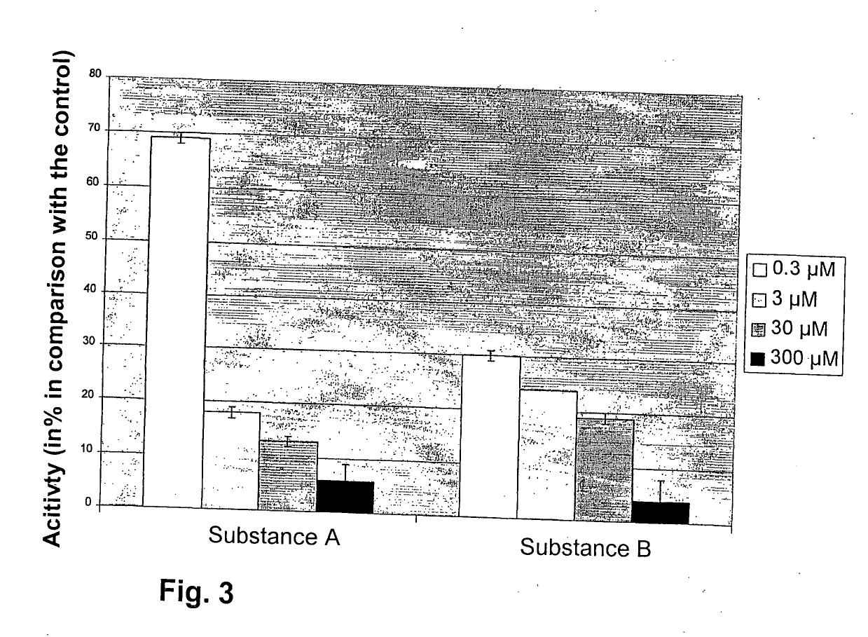

Inhibition of the ACCase from peach aphids Myzus persicae by two different substances A and B. The figure shows the respective ACCase activity at different inhibitor concentrations in comparison with the control (mixture without inhibitor not shown).

### REFERENCES

Abu-Elheiga L. et al. (1994): Human acetyl-CoA carboxylase: characterization, molecular cloning, and evidence for two isoformis. Proc. Natl. Acad. Sci. U.S.A. 92 (9), 4011-4015.

Altschul S F et al. (1997): Gapped BLAST and PSI-BLAST generation of protein database search programs. Nucleic Acids Res. 25: 3389-3402.

Bailey A et al. (1995): The ACC1 gene, encoding acetyl-CoA carboxylase, is essential for growth in Ustilago maydis. Mol. Gen. Genet. 249 (2), 191-201.

Devereux J et al. (1984): A comprehensive set of sequence analysis programs for the VAX. Nucleic Acids Res. 12: 387-395.

Goffeau A et al. (1996): Life with 6000 genes. Science 274 (5287).

Gronwald J W (1994): Herbicides inhibiting acetyl-CoA carboxylase. Biochem. Soc. Trans. 22 (3): 616-621).

Haselkorn R & Gornicki P (1999): Cyanobacterial and plant acetyl-CoA carboxylase, U.S. Pat. No. 5,972,644.

Jenkins A R et al. (1992): Maize Acetyl CoA Carboxylase encoding DNA clones, WO 93/11243 A1.

Ke J et al. (2000): Coordinate Regulation of the Nuclear and Plastidic Genes Coding for the Subunits of the Heteromeric Acetyl-Coenzyme A Carboxylase. Plant Physiology 122: 1057-1071.

Knowles J R (1989): The mechanism of biotin-dependent enzymes. Annu. Rev. Biochem. 58: 195-221.

Lottspeich, F., Zorbas H. (Ed.). 1998. Bioanalytik [Bioanalytics]. Spektrum Akademischer Verlag, Heidelberg, Berlin.Martinez-Zapater J. M. and Salina J., 1998 Humana Press ISBN 0-89603-391-0.

Mumberg D et al. (1995): Yeast vectors for the controlled expression of heterologous proteins in different genetic backgrounds. Gene 156, 119-122.

Munday M R & Hemingway C J (1999): The regulation of acetyl-CoA carboxylase—a potential for the action of hypolipidemic agents. Adv. Enzyme Regul. 39: 205-234.

Nauen R et al. (1996) Aphicidal activity of imidacloprid against a tobacco feeding strain of Myzus persicae (Homoptera: Aphididae) from Japan closely related to Myzus nicotinae and highly resistant to carbamates and organophosphates. Bull. Ent. Res. 86: 165-171

Puissant C & Houdebine L M (1990): An improvement of the single-step method of the RNA isolation by acid guanidinium thiocyanate-phenol-chloroform extraction. BioTechniques 8: 148-149.

Somers D A (1999): DNA encoding oat acetyl CoA carboxylase, WO 99/67367 A1.

Sutter M. et al. (1991): Acaricides containing soraphen A or soraphen B, EP-A412 937.

Vahlensieck H F et al. (1994): Identification of the yeast ACC1 gene product (acetyl-CoA carboxylase) as the target of the polyketide fungicide soraphen A. Curr. Genet. 25 (2): 95-100.

We claim:

1. A nucleic acid encompassing a sequence selected from
(a) the sequence as shown in SEQ ID NO: 1,
(b) the sequence as shown in Accession Number AAF59156,
(c) part-sequences of the sequences defined under (a) and (b) which are at least 14 base pairs in length,
(d) sequences which originate from insects or are derived therefrom and which hybridize with the sequences defined under (a) and (b) at a hybridization temperature of from 37° C. to 50° C.,
(e) sequences which originate from insects or are derived therefrom and which have at least 60% identity with the sequences defined under (a) and (b),
(f) sequences which are complementary to the sequence defined under (a) to (e), and
(g) sequences which, owing to the degeneracy of the genetic code, encode the same amino acid sequence as the sequences defined under (a) to (e).

2. A vector encompassing at least one nucleic acid as claimed in claim 1.

3. A vector as claimed in claim 2, characterized in that the nucleic acid molecule is linked operably to regulatory sequences which ensure the expression of the nucleic acid in pro- or eukaryotic cells.

4. A host cell encompassing a nucleic acid as claimed in claim 1 or a vector as claimed in claim 2 or 3.

5. A host cell as claimed in claim 4, characterized in that it is a pro- or eukaryotic cell.

6. A host cell as claimed in claim 5, characterized in that the prokaryotic cell is E.coli.

7. A host cell as claimed in claim 5, characterized in that the eukaryotic cell is a mammalian cell or an insect cell.

8. A polypeptide with a sequence selected from
a) the sequence with the biological activity of an acetyl-CoA carboxylase isolated from Myzus persicae,
b) the sequence as shown in SEQ ID NO: 2,
c) the sequence encoded by a nucleic acid of Accession Number AAF59156,
d) part-sequences of the sequences mentioned under a) to c) which retain the biological activity of an acetyl-CoA carboxylase,
e) sequences which originate from insects or are derived therefrom and which have at least 60% identity with the sequences mentioned under a) to d).

9. A method for preparing a polypeptide as claimed in claim 8, encompassing
(a) culturing a host cell as claimed in any of claims 4 to 7 under conditions which ensure the expression of a nucleic acid as claimed in claim 1, and
(b) obtaining the polypeptide from the cell or culture medium.

10. An antibody which reacts specifically with a polypeptide as claimed in claim 8.

11. The use of an insect polypeptide with the biological activity of an acetyl-CoA carboxylase for identifying insecticidally and/or acaricidally active compounds.

12. The use as claimed in claim 11, characterized in that the polypeptides are polypeptides as claimed in claim 8.

13. The use of nucleic acids which encode insect polypeptides with the biological activity of an acetyl-CoA carboxylase in methods for identifying modulators of these polypeptides.

14. The use of nucleic acids which encode insect polypeptides with the biological activity of an acetyl-CoA carboxylase for identifying substances which modify the expression of the polypeptides encoded by them.

15. The use as claimed in claim 13 or 14, characterized in that fragments of genomic DNA or cDNA are involved.

16. A method of finding a chemical compound which binds to an insect polypeptide with the biological activity of an acetyl-CoA carboxylase, encompassing the following steps:
(a) bringing an insect polypeptide with the biological activity of an acetyl-CoA carboxylase or a host cell comprising such a polypeptide into contact with a chemical compound or a mixture of chemical compounds under conditions which permit the interaction of one of these chemical compounds with the polypeptide, and
(b) identifying the chemical compound which specifically binds to the polypeptide.

17. A method of identifying substances which modulate the activity of acetyl-CoA carboxylase from insects and/or Acarina, characterized in that
a) the test substance is brought into contact with acetyl-CoA carboxylase under conditions which permit an interaction of the test substance with the acetyl-CoA carboxylase,
b) the interaction of the test substance which has taken place is detected by determining the ability of the acetyl-CoA carboxylase to catalyze the biotin-dependent carboxylation of acetyl-CoA, and
c) the ability of the acetyl-CoA carboxylase to catalyze the biotin-dependent carboxylation of acetyl-CoA in the presence of the test substance is compared with its ability to catalyze the biotin-dependent carboxylation of acetyl-CoA in the absence of a test substance.

18. A method of finding a compound which modifies the expression of insect polypeptides with the biological activity of an acetyl-CoA carboxylase, encompassing the following steps:
(a) bringing a host cell comprising a nucleic acid encoding an insect polypeptide with the biological activity of an acetyl-CoA carboxylase into contact with a chemical compound or a mixture of chemical compounds,
(b) determining the polypeptide concentration, and
(c) identifying the compound which specifically influences the expression of the polypeptide.

19. The use of compounds of the formula (I) 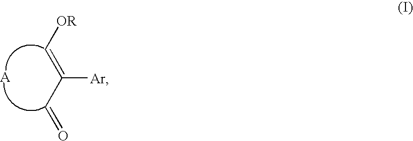
in which
Ar represents substituted aryl or hetaryl having at least one ortho-substituent,
R represents H or acyl radicals, and
A together with the linked C atoms forms an optionally substituted 5- or 6-membered carbo- or heterocycle, suitable heteroatoms being, for example, N, O and/or S,
as inhibitors of insect acetyl-CoA carboxylase.

21. The use of compounds of the formula (I) as claimed in claim 20 in methods as claimed in claims 17 and 18.

22. A modulator of acetyl-CoA carboxylase from insects and/or Acarina found by means of a method as claimed in claim 16 or 17.

23. An insecticidally and/or acaricidally active substance found by means of a method as claimed in claim 16 or 17.


SEQUENCE LISTING (2 sequences)

SEQ ID NO: 1 - DNA, 7047 bp, Drosophila melanogaster
  CDS (1)..(7047)
atgttgaagcgtcgcgccagcaagcgtttcgtacttgttgagtccggtgaagataatgccaacggctccggctcggcctcgggctctggctcgggatcaggagtgggaacggcggttataccccaatttgtggctgtggattgcgggcagaacgagagcaacaacaaccatgtcggcgagatgagtgccagcatcagcaatcacaatagctccaacaaccagtcgtcgccatcgctgctcagtgtgcccgtggtgggaaccctcaagccgagtatgtcgcgtggcacagggctgggccaggaccggcaccaggatcgcgacttccacatcgcaaccaccgaggagttcgtgaagcgctttggcggcacccgagtgatcaacaaggtcctgattgccaacaacggtatcgcggccgtcaagtgcatgcgatccatccggagatgggcgtacgaaatgtttaagaacgagcgggccattaggtttgtggtgatggtcactccggaggatctaaaggcgaatgccgaatacatcaagatggcggatcactatgtgcccgtgcccggcggatcgaacaacaacaactacgccaatgtcgagctcatcgtggacattgctcttcgcacccaagtgcaggccgtgtgggctggttggggtcatgcctccgagaacccgaagctgccggagctgcttcacaaagagggtctggtgttccttggccctccggaacgtgccatgtgggcgctgggcgacaaggtggcctcctctattgtggcccaaacggccgagattcccaccctgccgtggtccggttcggacctgaaggcccagtacagtggcaaaaagatcaagatttccagtgagctcttcgcccgaggttgtgtgaccaatgtggaacagggtctggccgcagttaacaagattggcttccccgtaatgatcaaggcctcggaaggaggtggtggcaagggtattcgccgcgtggacaccactgaggagttccccggcctgttccgccaggttcaagctgaggtgcccggctcaccgattttcgtgatgaagctggcccgcggagctcgccacttggaggtgcaactgttggcagatcagtacggcaatgccattagcttgttcggccgtgactgctccatccagcgtcgtcatcagaaaattattgaggaagctcctgccatcgtggcccagccagaggtgttcgaggacatggagaaggccgccgtgcggttggccaagatggtgggttacgtcagcgcgggaaccgtggagtacctatatgatccggagggtcgctacttcttcctggagctgaacccacgtttgcaggtggagcatccgtgtacggagatggtggccgatgtaaatcttccagctgctcagctgcagattggaatgggaattcccctttaccggctcaaggacatccgtctgctgtacggagagtctccctggggctcctcagtcattgacttcgaaaatccaccgaacaaaccgcgtccctccggacatgttatcgctgctcgtatcacctcagagaaccccgacgagggctttaagcccagttctggaaccgttcaggagcttaacttccggtcgagcaaaaatgtgtggggctacttcagtgtggctgccagtggaggattgcacgagttcgcggattcacagtttgggcattgtttctcctggggcgagaaccgtcaacaggctcgagagaacctggtgattgccctgaaggagctgtcaattcgaggtgatttccgaaccacagtggaatacttgatcactctgctcgaaacgaatcggttcctcgacaacagcatcgacaccgcctggctagatgccttgatcgcagagcgtgtgcaatccgagaagccggatatcctgttgggcgtaatgtgcggatcgctgcacatcgcagatcgtcaaattactgagagcttttccagcttccaaacctctctggagaaaggtcagatccaagcagcgaacacgctgacgaacgtggtggatgttgagctaatcaacgatggcatccgttacaaggtgcaggccgccaagagcggagccaactcgtacttcctgctgatgaacagctcgtttaaggagatcgaggtgcaccgcctctccgacggaggcttgctcatctctttggagggcgcctcctacaccacgtacatgaaggaggaggtggatcgctaccgcattgtgattggcaaccagacatgtgtctttgaaaaggagaacgatccatcgctgttgcgcagtccgtctgcgggaaagctcatcaacatgattgtggaagatggcgctcatgtaagcaagggccaggcctatgctgagattgaggtgatgaagatggtgatgaccctgacgtcccaggaggcaggcacagtgacatttgtgcgtcgaccaggagctgttctagatgcaggatcccttttgggccacttggagctggacgatccatcgctggtgacgaaagcgcagcccttcaagggacagttcctgcagccagagaacgcaccggtacccgagaaactaaacagggtgcacaatacttacaagagtatccttgaaaacacactggctggttactgcctgccagaaccgttcaatgcacagcgactcagagacatcatcgaaaaattcatgcaaagcttgcgtgatccctcgttgccgttgttggagctgcaagaagttatcgcctccatctctggtcgcatacccatatccgtggagaagaagatccggaaactgatgacgctgtacgagcgaaacataactagtgtcctggcccaattcccctcgcagcagatcgccagtgttattgacagccatgcggccacgctgcagaagcgcgctgaccgtgatgtcttcttcctgaccacccagagcattgtgcagctggtgcagcgctataggaacggaatccgcggcagaatgaaggccgccgttcatgagctgttgcgtcagtactacgatgtagagtcgcagttccagtatggacactacgacaaatgcgtgggactggtgcgagagcacaacaaggacgacatgcagacggtggtcaacaccatcttctcgcactctcaggtggccaagaagaatctgctggtcactctgctcattgatcacctgtgggccaacgaacctggactaacggacgaattggccaacacgctaagtgaattgacctctttgaatcgagctgagcactctagggttgccctgcggtcccgccaagttctgatcgctgcccaccagccggcttatgagctgcgccacaaccaaatggagtcgatctttctctccgccgttgacatgtacggtcatgacttccaccccgagaacctgcagcgcctgattctgtcggagacctcaatctttgacatcctgcacgacttcttctaccactctaaccgggcagtgtgcaatgctgctctggaagtctatgtgaggagagcttacacatcctatgagctgacctgcttgcagcatttggaactctccggtggcctgccgctggtgcacttccagttcctcctccccacagctcacccgaacagactgttctcgcgcatgtcctcccccgatggattggatcaggcagcggcagagtctttgggaaactcattcgtgcgcaccggagcgattgcagcctttgactccttcgaacactttgagatgtactcggacgagattctggatctgctcgaagacttcgtctcgccagccatggttaatgccaaggtcctggaagccgtagaggcagcggattctatctcggacagccgacacagcacctcgatcaatgtgtcgttgtcggatcccgtaacccgggcgaatgctgccgaggaggccaagtccaccgaaccgattcacattgttagtgtggctgtgagagaaacgggggagttggatgacctgcaaatggcccaaatctttggaaattattgccaagagcataacgaggagctcttccagcgacgcattcgtaggattacatttgctgctctgaagaagcggcaattccccaagttctttacgttcagagccagagataagttcacggaggatcgtatttaccggcatctggagccagcatctgctttccatctggagctgaaccgcatgaagacgtacgatctggaggctctgcccacggctaaccaaaagatgcacctgtaccttggcaaggccaaggtttcgaaaggtcaagaggtcacggactaccgcttcttcattcgctcgatcatccgtcattcggatctgattaccaaggaagcctctttcgagtatctgcaaaacgaaggagagcgtgtgctcctggaggccatggatgagctggaggtggcattctcgcatccgcacgccaaacgcacggactgcaaccacatcttcctgaactttgtgcccaccgtcatcatggatccggctaagatcgaggaatctgtaacaaagatgattatgcgatatggtccacgtctttggaagctgcgtgtactgcaggctgagctcaagatggtcatccgccagtcaccacagtcacccactcaggcagtgcgtctgtgcattgcaaatgactccggctacttcctggatatttcgatgtataccgaacaaacagaaccagagacaggaatcattaagtttaaggcctacggtgagaagcagggatctctgcacggacatcccatttcgacgccctacatgaccaaggacttcctgcagcagaaacgtttccaggcgcagtccaatggtaccacctatgtctatgatgtgcccgacatgttccgccagatgaccgagcgtcactggagagaattctccaaggctcgtcccaccgtggacattcgcactcccgacaagattttgatcgagtgcaaggagctggtcctcgagggcgacaaccttgtagagatgcagcgtctgcccggcgaaaacaattgcggcatggtggcttggcgcattgtcttggctactccggaatatccgaatggccgcgagatcattgttatagccaacgatctcacctacttgattggttcctttggaattaaggaggacgttctctttgccaaggcttcccaattggctcgccaactcaaagtaccgaggatatacatctccgttaacagcggtgcccgcataggacttgctgaggaggttaaagctatgttcaagatcgcatgggaggatccagaggagccagataagggcttcaagtacctctacttgagcaccgaggactacgcccaggtggccaacctgaactcggtgagggctatcctgatcgaggacgagggcgagcagcgttacaagattaccgacatcatcggcaaggacgatggtctgggcgtggagaatctgcgttacgccggcttgattgccggtgaaacgtcgcaggcctacgaggagattgttactatcgctatggttacctgccgtaccattggcattggatcctatgtggtgcgcctgggtcagcgcgttatccagatcgataattcacacattatactcactggctatgctgcgcttaacaagctgcttggacgcaaggtgtatgcctctaataatcagttgggtggcacacagatcatgtttaacaacggagtcacccacaaaacagaggccatcgacttggacggtgtctacaccatcctcgactggctctcgtacatccccgcgtacatcggttgtgacctgcccattgttttgcccaacgatcgtatcgaacgccctgtcgacttcatgcccaccaagtcgccctacgatccgcgctggatgctgggtggccgtgtgaatcccgtgaacgctaatgactgggagaacggattctttgatcgcgactcgtggagcgaaatcatggcctcgtgggccaagacagtggtcactggtcgcgcacgtctaggcggtgtccccgtgggcgtaatagccgttgagacccgcaccgtagaagtggagatgcccgccgatcctgccaatctcgattcggaagccaagaccctgcagcaggcaggtcaggtgtggtaccccgactcctcgtacaaaacggcacaagcgatcaaagattttggacgagaggagttgccgctgattgttttcgcaaattggcgaggcttctccggtggcatgaaggacatgtacgagcaaatcgtcaagttcggagcatacattgtcgacggcctgcgggagtacaagaagcctgtgctcatctacctgccgcccaacgccgagctgcgaggtggagcctgggccgtgttggattccctcattaacccgcgctacatggaaacgtatgccgatccggaggccagaggaggagttctcgagccggagggcattgtggaaataaagtacaaagagaaggacctggtcaagacgattcaccgcttggatccgaccaccattgcgctgaaaaaggagctcgatgaggcaaatgcgtctggcgacaaggtcagggctgctcaggtggacgaaaagatcaaggcccgcatcgctgtgctaatgcacgtctaccacacggtagcagttcactttgccgacctgcacgacacgccggagcgaatgctagagaaggagtgtatcagtgagattgtgccttggcgcgattcccgccgctggctgtactggcgtctgcgacgtctcctgttggaggacgcatatattaagaagatcctgcgcgctcaggacaacctctccgtgggtcaggccaagcagatgctgcgtcgatggctggtagaggagaagggtgccacagaggcttatctgtgggacaaaaacgaggagatggtgtcttggtatgaggagcagatcaatgccgaatctattgtttcccgcaacgtgaactccgtgagacgggatgccattatttctaccatttcgaaaatgctcgaggactgtcccgacgtagcgctggacgctgttgtgggtctttgccaaggtctgacgccagtgaatcgaggcgtggtcgtacgcacattagcccagatgcagctgaatgaggagacctctaacagcaaccagggatga

SEQ ID NO: 2 - protein, 2348 aa, Drosophila melanogaster
MLKRRASKRFVLVESGEDNANGSGSASGSGSGSGVGTAVIPQFVAVDCGQNESNNNHVGEMSASISNHNSSNNQSSPSLLSVPVVGTLKPSMSRGTGLGQDRHQDRDFHIATTEEFVKRFGGTRVINKVLIANNGIAAVKCMRSIRRWAYEMFKNERAIRFVVMVTPEDLKANAEYIKMADHYVPVPGGSNNNNYANVELIVDIALRTQVQAVWAGWGHASENPKLPELLHKEGLVFLGPPERAMWALGDKVASSIVAQTAEIPTLPWSGSDLKAQYSGKKIKISSELFARGCVTNVEQGLAAVNKIGFPVMIKASEGGGGKGIRRVDTTEEFPGLFRQVQAEVPGSPIFVMKLARGARHLEVQLLADQYGNAISLFGRDCSIQRRHQKIIEEAPAIVAQPEVFEDMEKAAVRLAKMVGYVSAGTVEYLYDPEGRYFFLELNPRLQVEHPCTEMVADVNLPAAQLQIGMGIPLYRLKDIRLLYGESPWGSSVIDFENPPNKPRPSGHVIAARITSENPDEGFKPSSGTVQELNFRSSKNVWGYFSVAASGGLHEFADSQFGHCFSWGENRQQARENLVIALKELSIRGDFRTTVEYLITLLETNRFLDNSIDTAWLDALIAERVQSEKPDILLGVMCGSLHIADRQITESFSSFQTSLEKGQIQAANTLTNVVDVELINDGIRYKVQAAKSGANSYFLLMNSSFKEIEVHRLSDGGLLISLEGASYTTYMKEEVDRYRIVIGNQTCVFEKENDPSLLRSPSAGKLINMIVEDGAHVSKGQAYAEIEVMKMVMTLTSQEAGTVTFVRRPGAVLDAGSLLGHLELDDPSLVTKAQPFKGQFLQPENAPVPEKLNRVHNTYKSILENTLAGYCLPEPFNAQRLRDIIEKFMQSLRDPSLPLLELQEVIASISGRIPISVEKKIRKLMTLYERNITSVLAQFPSQQIASVIDSHAATLQKRADRDVFFLTTQSIVQLVQRYRNGIRGRMKAAVHELLRQYYDVESQFQYGHYDKCVGLVREHNKDDMQTVVNTIFSHSQVAKKNLLVTLLIDHLWANEPGLTDELANTLSELTSLNRAEHSRVALRSRQVLIAAHQPAYELRHNQMESIFLSAVDMYGHDFHPENLQRLILSETSIFDILHDFFYHSNRAVCNAALEVYVRRAYTSYELTCLQHLELSGGLPLVHFQFLLPTAHPNRLFSRMSSPDGLDQAAAESLGNSFVRTGAIAAFDSFEHFEMYSDEILDLLEDFVSPAMVNAKVLEAVEAADSISDSRHSTSINVSLSDPVTRANAAEEAKSTEPIHIVSVAVRETGELDDLQMAQIFGNYCQEHNEELFQRRIRRITFAALKKRQFPKFFTFRARDKFTEDRIYRHLEPASAFHLELNRMKTYDLEALPTANQKMHLYLGKAKVSKGQEVTDYRFFIRSIIRHSDLITKEASFEYLQNEGERVLLEAMDELEVAFSHPHAKRTDCNHIFLNFVPTVIMDPAKIEESVTKMIMRYGPRLWKLRVLQAELKMVIRQSPQSPTQAVRLCIANDSGYFLDISMYTEQTEPETGIIKFKAYGEKQGSLHGHPISTPYMTKDFLQQKRFQAQSNGTTYVYDVPDMFRQMTERHWREFSKARPTVDIRTPDKILIECKELVLEGDNLVEMQRLPGENNCGMVAWRIVLATPEYPNGREIIVIANDLTYLIGSFGIKEDVLFAKASQLARQLKVPRIYISVNSGARIGLAEEVKAMFKIAWEDPEEPDKGFKYLYLSTEDYAQVANLNSVRAILIEDEGEQRYKITDIIGKDDGLGVENLRYAGLIAGETSQAYEEIVTIAMVTCRTIGIGSYVVRLGQRVIQIDNSHIILTGYAALNKLLGRKVYASNNQLGGTQIMFNNGVTHKTEAIDLDGVYTILDWLSYIPAYIGCDLPIVLPNDRIERPVDFMPTKSPYDPRWMLGGRVNPVNANDWENGFFDRDSWSEIMASWAKTVVTGRARLGGVPVGVIAVETRTVEVEMPADPANLDSEAKTLQQAGQVWYPDSSYKTAQAIKDFGREELPLIVFANWRGFSGGMKDMYEQIVKFGAYIVDGLREYKKPVLIYLPPNAELRGGAWAVLDSLINPRYMETYADPEARGGVLEPEGIVEIKYKEKDLVKTIHRLDPTTIALKKELDEANASGDKVRAAQVDEKIKARIAVLMHVYHTVAVHFADLHDTPERMLEKECISEIVPWRDSRRWLYWRLRRLLLEDAYIKKILRAQDNLSVGQAKQMLRRWLVEEKGATEAYLWDKNEEMVSWYEEQINAESIVSRNVNSVRRDAIISTISKMLEDCPDVALDAVVGLCQGLTPVNRGVVVRTLAQMQLNEETSNSNQG